(19) United States
(12) Patent Application Publication
(10) Pub. No.: US 2007/0293868 A1
(43) Pub. Date: Dec. 20, 2007

(54) Ligament-Tentioning Device, Section Template and Osteotomy Method

(76) Inventors: Daniel Delfosse, Jegenstorf (CH); Walter Supper, Grenchen (CH); Christoph Fankhauser, Solothurn (CH)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/665,789

(22) PCT Filed: Oct. 18, 2005
(86) PCT No.: PCT/EP05/11203

§ 371 Date: Aug. 1, 2007

(30) Foreign Application Priority Data
Oct. 19, 2004 (DE) 10 2004 050 913.1

(51) Int. Cl. A61B 17/58; A61B 17/04

(52) U.S. Cl. 606/88; 606/144; 606/148

(57) ABSTRACT

A ligament-tensioning device (1) for preparing the implantation of an articulated implant consists of a base body (5) comprising first claws (6, 6′) provided with first bearing surfaces (7, 7′) applied to a first bone part (33) and second claws (13, 13′) provided with a second bearing surfaces (10, 10′) applied to a second bone part (34). The claws (6, 6′; 13, 13′) are displaceable with respect to each other with the aid of parallel-displacement devices (12, 12′). In addition, the invention provides for a first scale (31) disposed on the base body (5) of the ligament-tensioning device (1) and second medial and lateral scales (32, 32′) corresponding to the first scale (31), wherein the second scales (32, 32′) are disposed on a guiding body (3) which is movably mounted with respect to said base body (5) by means of the parallel-displacement devices (12, 12). The scales (31, 32; 31, 32′) can be brought into congruence in such a way that the height of an implant insertable into the treatable joint is separately medially and laterally presettable.

The invention relates to a ligament-tensioning device for joints of the human or animal body, a section template suitable for use on a joint prepared by means of the ligament-tensioning device, as well as an osteotomy method for these joints using the ligament-tensioning device according to the invention and the section template according to the invention.

From WO 03/084412 A1 a ligament-tensioning device for preparing for the implantation of a joint implant is known, having a basic body, which comprises a first claw with a distal seating surface, which rests against a first bone, and a second claw, which rests with a proximal seating surface against a second bone. The second claw is displaceable parallel to the first claw. A section template is positionable on holding devices of the basic body of the ligament-tensioning device.

From WO 00/78225 A1, moreover, a ligament-tensioning device for non-spherical joints is known. The device described therein for tensioning ligaments of non-spherical joints in the human or animal body comprises a prismatic, cylindrical or plate-shaped basic body having a right claw and a left claw, which have first contact surfaces in one plane and are therefore bringable parallel into contact with the joint-side surface of a first bone adjoining a non-spherical joint, as well as a right handle and a left handle, a right tension lever and a left tension lever having second contact surfaces, which are disposed parallel to the first contact surfaces, wherein between the respective contact surfaces of the right tension lever and the right claw a tensioning width Y and between the respective contact surfaces of the left tension lever and the left claw the same or a different tensioning width X is adjustable. The second contact surfaces are bringable into contact with the joint-side surface of a second bone adjoining the joint. The device further comprises a right control lever and a left control lever, which simultaneously with holding the device by one hand each on the appropriate handle are actuable individually using in each case the same hand, and a right parallel displacement device and a left parallel displacement device, which are each operable by the appropriate control lever and are each connected in such a way to a tension lever that, upon a movement of the control levers, the tensioning widths X and Y are adjustable independently of one another. The parallel displacement devices take the form of four-bar lever mechanisms.

The drawback of the ligament-tensioning devices known from the previously cited printed publications is in particular that the provision of section planes on a diseased joint for the introduction of a prosthesis requires further tools, which are positioned independently of the ligament-tensioning device against the joint and hence do not allow accurate positioning and alignment or reproducible, accurate section guidance.

The underlying object of the invention is accordingly to provide a ligament-tensioning device and a method of flexing the capsular ligament structures of a joint, which is to be replaced, by means of a parallel spreading motion and in so doing allow adjustment of a preadjustable, re-adjustable and checkable resection height for the medial and the lateral side separately of one another.

A further object of the invention is to indicate a section template that offers an economical and accurate section guide for various sizes of femur.

The object is achieved with regard to the ligament-tensioning device by the features of claim 1, with regard to the method by the features of claim 10 and with regard to the section template by the features of claim 16.

Further advantageous developments of the invention are characterized in the sub-claims.

There now follows a detailed description of the invention with reference to partially diagrammatic representations of the preparation for replacement of a human knee joint.

The drawings show:

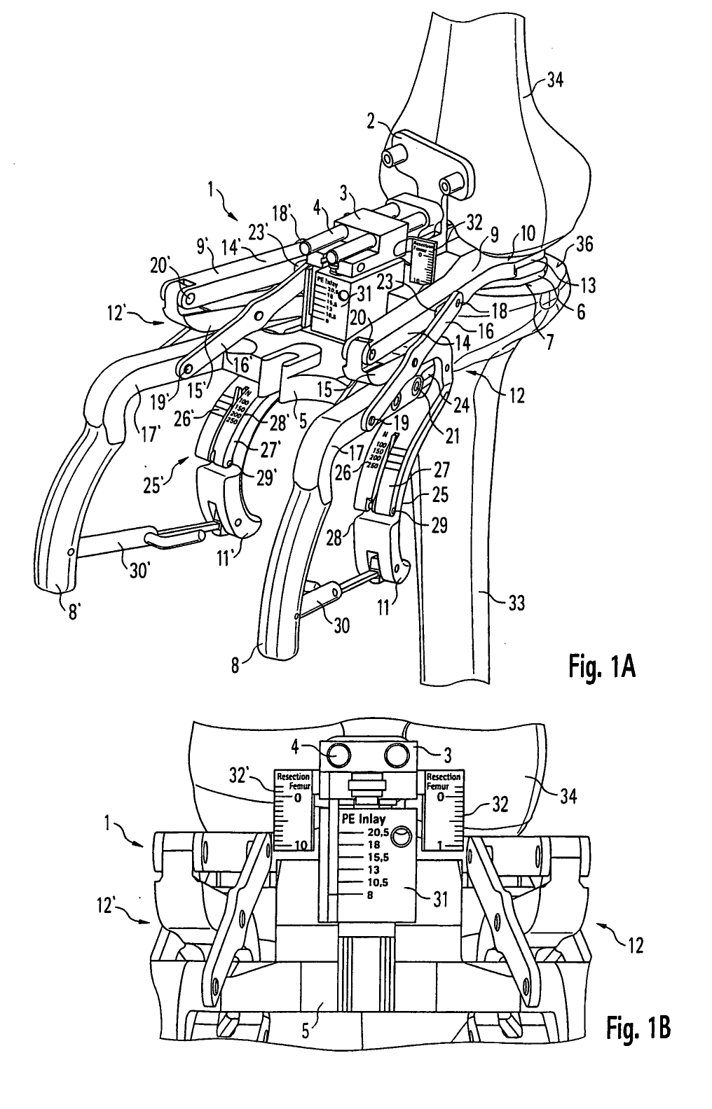

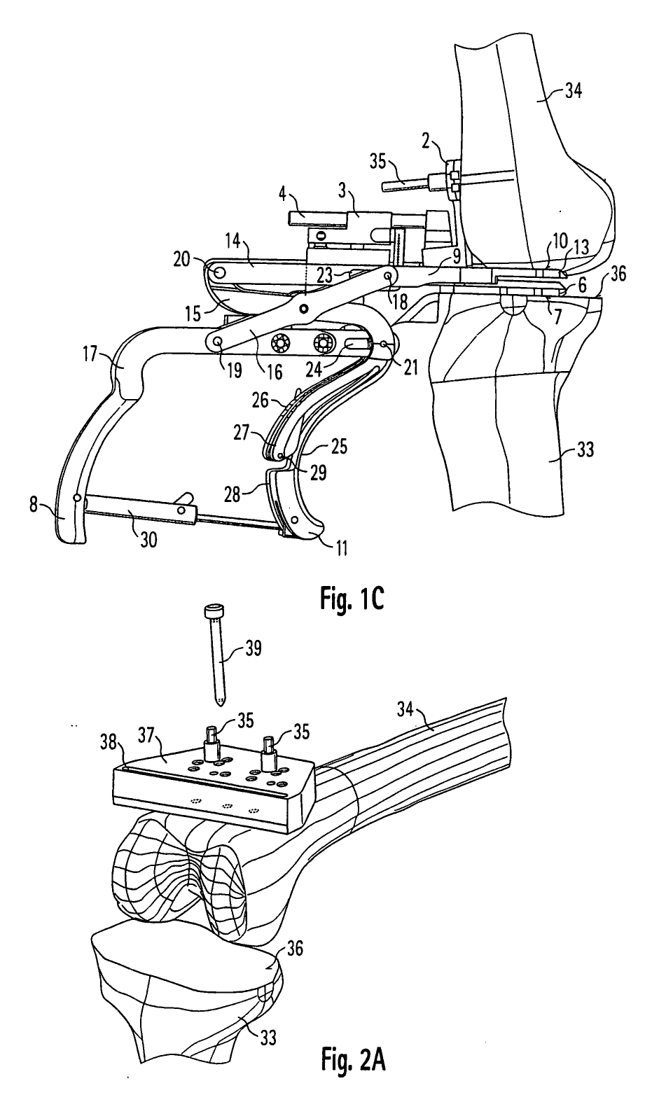

FIG. 1A a diagrammatic, perspective view of a ligament-tensioning device according to the invention with a drilling template,

FIG. 1B an enlarged representation of the ligament-tensioning device represented in FIG. 1A viewed in posterior direction,

FIG. 1C a diagrammatic, perspective view of a ligament-tensioning device according to the invention viewed in medial direction,

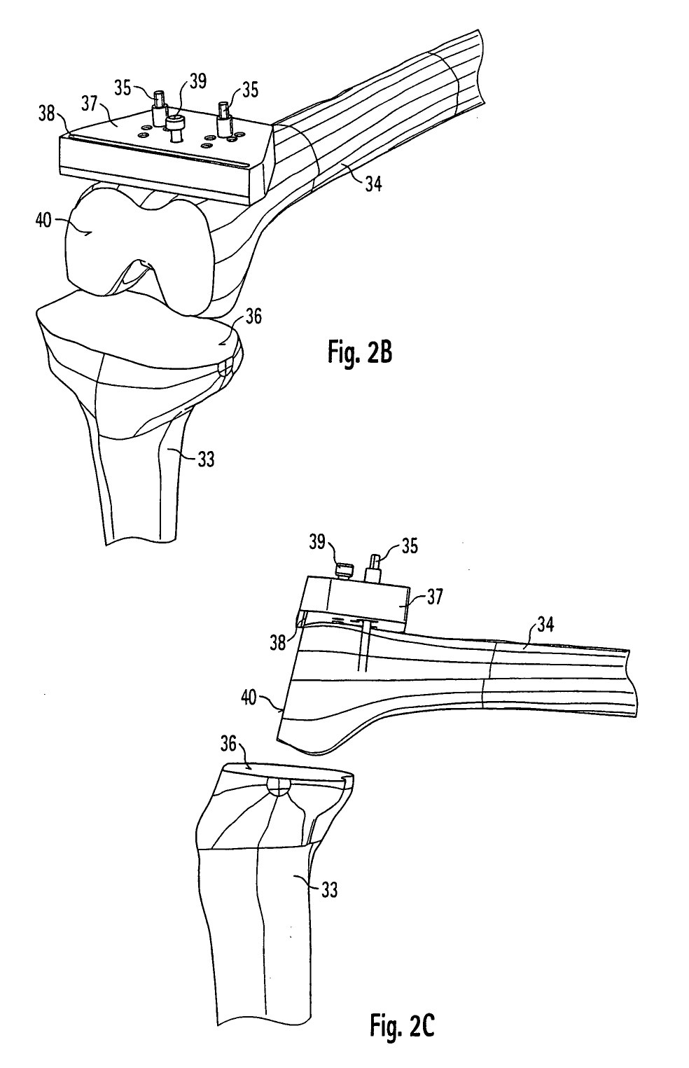

FIGS. 2A-C diagrammatic, perspective representations of a distal femoral osteotomy using a section template,

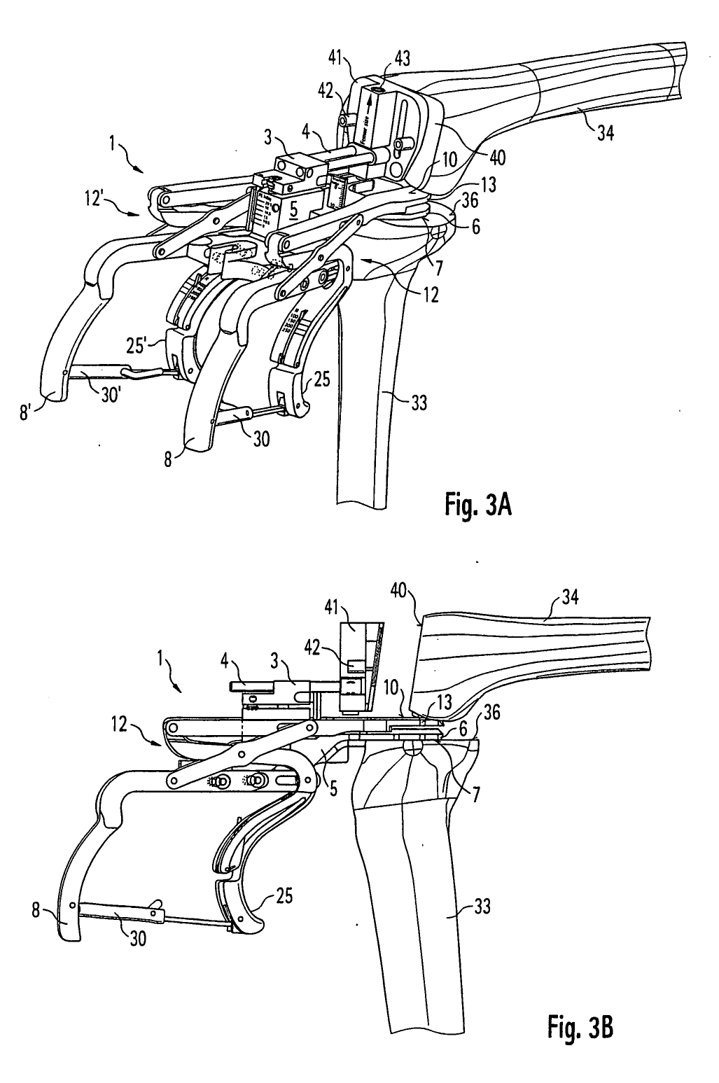

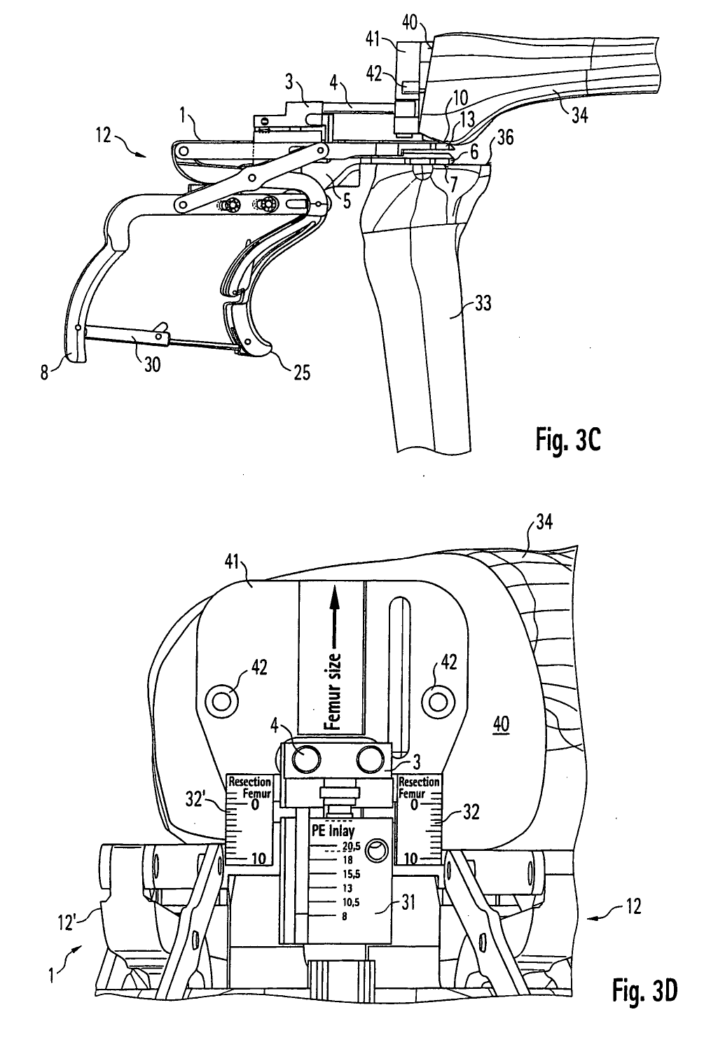

FIGS. 3A-D diagrammatic, perspective representations of the attachment of a drilling template for preparing the drill holes for the section template according to the invention,

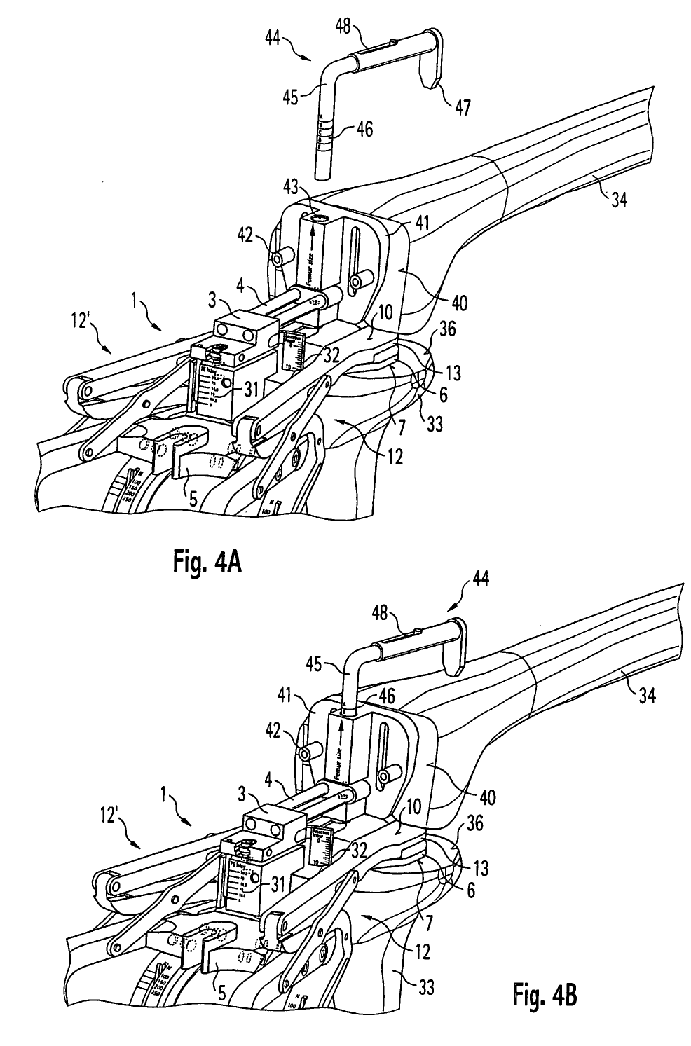

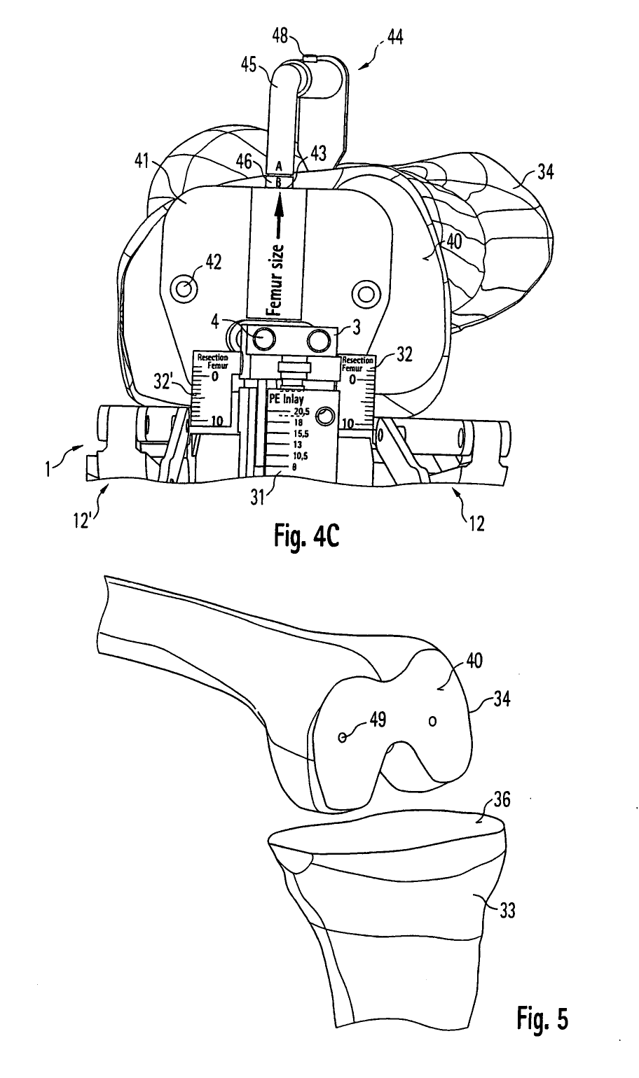

FIGS. 4A-C diagrammatic, perspective representations of the attachment of a feeler template for determining the femur size,

FIG. 5 a diagrammatic, perspective representation of the knee joint prepared for the attachment of the section template designed in accordance with the invention, and

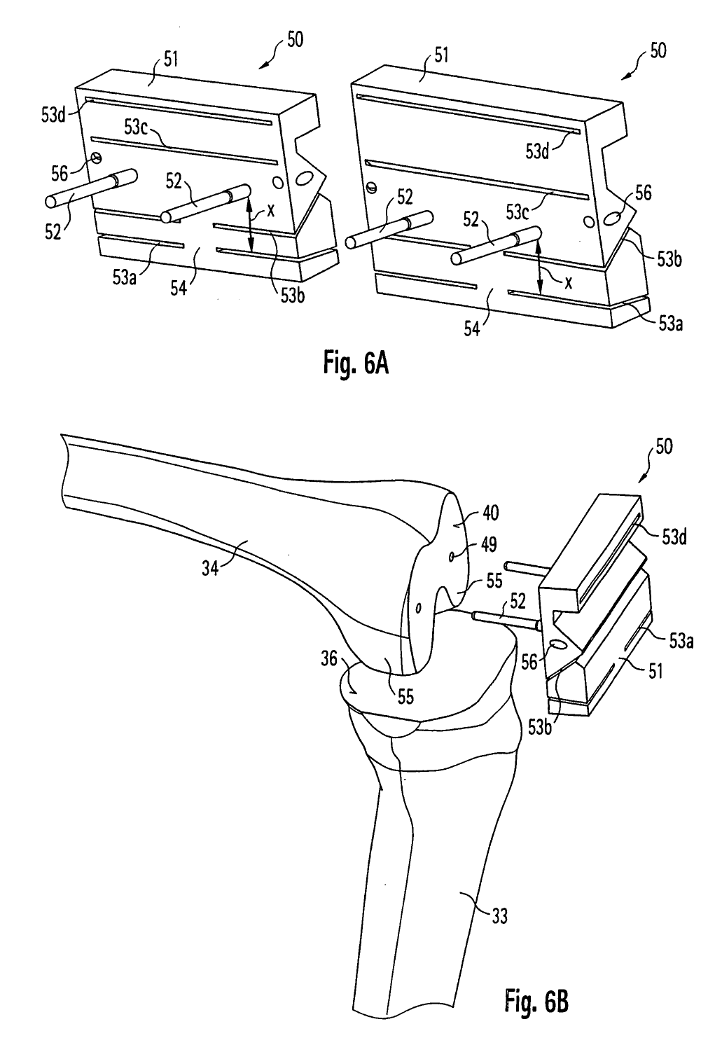

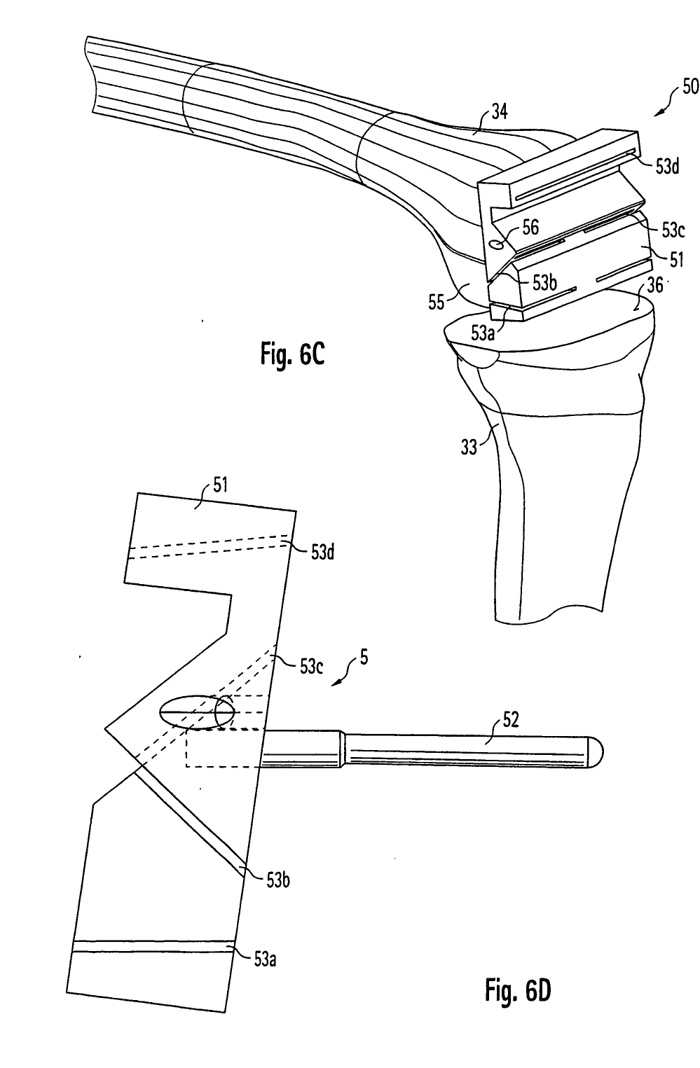

FIGS. 6A-D diagrammatic, perspective representations of the section template according to the invention ex situ and in situ.

FIG. 1A shows in a diagrammatic, perspective overall view a ligament-tensioning device 1 comprising a basic body 5, on which a guide body 3 with guide rods 4 is disposed. Various drilling templates 2 for the preparation of resections in the region of a joint that is to be replaced, for example a knee joint, are mountable and displaceable on the guide rods 4.

The ligament-tensioning device 1, for safe introduction of the spreading force into a first bone part 33, comprises first claws 6, 6′ (not visible in FIG. 1A) having first contact surfaces 7, 7′ (likewise not visible in FIG. 1A), which in the case of the knee joint rest on the tibia 33 (head of the shin-bone). Opposite the first claws 6, 6′ there are provided in a corresponding manner on the basic body 5 handles 8, 8′, which allow in each case single-handed holding and tensioning of the ligament-tensioning device 1. Likewise in a corresponding manner to the arrangement of the first claws 6, 6′ and above these claws, the ligament-tensioning device 1 comprises tension levers 9, 9′, which are supported by their second contact surfaces 10, 10′, which are formed on second claws 13, 13′ (likewise not visible in FIG. 1A), on a second, opposite bone part 34 of the joint to be treated, in the case of the knee joint the femur 34. The spreading action is produced by actuating the handles 8, 8′ together with in each case a control lever 11, 11′ for a medial or lateral joint component separately or jointly.

Parallel displacement devices 12, 12′ allow, with regard to the contact surfaces 7, 7′ and 10, 10′, a parallel displacement of the second claws 13, 13′ having the contact surfaces 10, 10′ relative to the first claws 6, 6′ having the contact surfaces 7, 7′. The second claws 13, 13′ are in said case workingly connected to the tension levers 9, 9′.

The parallel displacement devices 12, 12′ are designed as a four-bar linkage in the form of intersecting rods and comprise in each case four levers 14, 14′, 15, 15′, 16, 16′, 17, 17′, wherein tension-lever-side levers 14, 14′ and basic-body-side levers 17, 17′ are disposed parallel to one another, while the levers 15 and 16 as well as 15′ and 16′ intersect. The four levers 14, 15, 16, 17 and 14′, 15′, 16′, 17′ are connected to one another by means of five axles 18, 19, 20, 21, 22 and 18′, 19′, 20′, 21′, 22′ respectively. Two of the axles 18, 19 and/or 18′, 19′ are mounted displaceably in the parallel levers 14, 17 and/or 14′, 17′ in oblong holes 23, 23′, 24, 24′ extending parallel to the contact surfaces 7, 7′, 10, 10′. This development of the parallel displacement devices 12, 12′ allows the tension-lever-side levers 14, 14′ and the basic-body-side levers 17, 17′ to be movable parallel to one another and/or apart from one another.

The lengths of the levers 14, 14′, 15, 15′, 16, 16′, 17, 17′ are so selected that, given any desired tensioning width X between the contact surfaces 7, 7′ on the first claws 6, 6′ and the contact surfaces 10, 10′ on the second claws 13, 13′, which may be for example between 5 mm and 40 mm, there is a constant conversion ratio of 1:1 between the tensioning force applied manually at the handles 8, 8′ and the control levers 11, 11′ and the distraction force exerted on the bones adjoining the joint.

The magnitude of the spreading force is readable at force indicators 25, 25′ having scales 26, 26′ and movable indicating levers 27, 27′. The indicating levers 27, 27′ are moved as a result of the longitudinal bending of the control lever parts 28, 28′, which are bendable by means of a manually applied tensioning force, relative to the other indicating levers 27, 27′, which are arranged in a fork-like manner and are not loaded with this tensioning force. If by means of the tensioning force the indicating lever 27, 27′ and the control lever parts 28, 28′ are moved relative to one another, the indicating levers 27, 27′ rotate about axes of rotation 29, 29′, with the result that the manually applied tensioning force is indicated at the scales 26, 26′ by the indicating levers 27, 27′.

Locking devices 30, 30′ may moreover be provided between the handles 8, 8′ and the control levers 11, 11′ and allow the ligament-tensioning device 1 to be locked in a specific position.

The basic body 5 of the ligament-tensioning device 1 has a first scale 31, which corresponds with the second scales 32, 32′. The scales 31, 32 and 32′ (likewise not visible in FIG. 1A) indicate the planned resection height medially and laterally on the bone, e.g. on the femur 34 taking account of the ligament situation and with the tibia 33 already resectioned, thereby allowing measurement of the medial and lateral resection heights prior to the posterior and anterior resection. By the selection of the femoral resection height an optimum reproduction of the physiological articular plane is possible. The precise function of the ligament-tensioning device 1 is outlined in detail in the following drawings and in the accompanying description.

In these drawings, for the sake of clarity, the reference characters of components that are not relevant to the invention are not repeated. Only some parts that are of assistance for orientation purposes are denoted. There is likewise no repeat description of the appropriate components in the description that follows.

FIG. 1B shows in a diagrammatic view in posterior direction a plan view of the ligament-tensioning device 1. Here, it is possible to see in particular the scales 31 and 32, 32′, which according to the invention, as already mentioned, indicate the planned resection heights medially and laterally on the femur 34 while taking account of the ligament situation and allow measurement of the resection heights medially and laterally in order to determine the correct resection heights perpendicular thereto, i.e. in posterior and anterior direction. This allows an optimum reproduction of the physiological articular plane since both the mediolateral direction and the anteroposterior direction are included in the measurement and hence in the section guide of the resection. The special arrangement of the scales 31, 32, 32′ additionally makes it possible to check a rotational movement of the femur 34 that occurs during flexion and extension of the joint and, if not correctly included, may lead to problems in the replacement joint.

The scales 31 and 32 as well as 31 and 32′ correspond in each case to one another. As the ligament-tensioning device 1 is equipped with two mutually independently operating parallel displacement devices 12, 12′ that are actuable independently of one another, it is therefore possible to set different widths of the knee joint gap and/or of the inlay medially and laterally, so that optimum account may be taken of the ligament situation of the joint.

Generally, a replacement joint comprises a plurality of components which, depending on the condition of the joint, are fitted into one or into both bone parts 33, 34. In the case of total joint replacement, an endoprosthesis is necessary, which may additionally comprise an inlay that lies between the prosthetic parts and, in the case of the knee joint, performs the function of the menisci. For satisfactory patient care it is important to correctly determine the height of the inlay and, as a preparatory measure, the resection height of the bones 33, 34 involved.

In this case, it is helpful both to be able to adjust the height of the knee joint gap in flexion and extension in discrete values analogously to the available inlay sizes and to have the option of using an infinite adjustment of the flexion- and extension gap height that allows knee-specific over-/under-corrections of the knee joint gap by means of a continuous bone resection. It is further desirable to be able to determine reliably the optimum anterior alignment of the femur component of the endoprosthesis that determines the transition between the implant component relative to the anterior cortex, i.e. to the anterior projection.

FIG. 1C shows in a side view the situation according to FIGS. 1A and 1B. The drilling template 2 is in this case already in contact with the femur 34. Two drill holes are introduced into the femur 34 by means of a drill and receive a section template, in the manner described further below.

The preparatory steps needed for correct section guidance are explained by means of the following drawings and the accompanying description.

As is not shown in detail, the tibia 33 is prepared by means of conventional resection methods so as to form a transverse surface 36, against which the claws 6, 6′ of the ligament-tensioning device 1 rest.

The procedure for determining the correct inlay thickness and/or the resection height as well as the preparations for the resection are described below.

Firstly, in extension, i.e. in the stretched state of the knee joint, the ligament-tensioning device 1 is inserted into the knee joint gap between tibia 33 and femur 34. The drilling template 2 for introducing the drill holes for the section template that is used for a first, distal femoral resection has already been mounted onto the ligament-tensioning device 1.

Then, by means of actuation of the ligament-tensioning device 1 in extension position the ligaments are distracted under a selectable force. The force is read off and adjusted at the scales 26, 26′ of the force indicators 25, 25′. The values read off at the scales 31, 32 and 32′ give the resection height of the first, distal femoral resection and/or the thickness of the inlay that is to be inserted later between the prosthetic components. The separate adjustability for the medial and the lateral side may lead to the occurrence of different values on the scales 32, 32′ that correspond to a rotation of the femur 34. The rotation is preferably 1° per millimetre of the scales 32, 32′.

If the distance to be read off at the scales 31, 32, 32′ is too great, a follow-up resection of the tibia 33 has to be performed. If the distance is too small, a larger inlay size has to be selected.

Then, as is evident from FIG. 1C, two drill holes are drilled into the femur 34 by means of a drill. Pins 35 are inserted into the drill holes. The ligament-tensioning device 1 is then relaxed and removed from the knee joint gap.

FIGS. 2A to 2C show in various views the attachment of the section template 37 that is required for the distal femoral resection.

In flexion, as is evident from FIGS. 2A to 2C, the joint is bent, i.e. situated at an angle, and the section template 37 is mounted onto the pins 35. To prevent displacement of the section template 37, it is fixed to the femur 34 by means of a fixation nail 39. The section template 37 has a saw blade guide 38, by means of which a saw blade is guided during the resection.

In FIGS. 2B and 2C the resection has already been completed, thereby resulting likewise in the formation on the femur 34 of a transversely oriented surface 40, which in extension is oriented parallel to the transverse surface 36 of the tibia 33.

FIGS. 3A to 3D illustrate the next step in preparation for the second femoral resection.

As is shown in FIG. 3A in a perspective overall view, the ligament-tensioning device 1 is positioned once more against the joint, which is still in flexion. The drilling template 2 is replaced by a second drilling template 41, which is mounted in an identical manner to the drilling template 2 on the ligament-tensioning device 1.

FIGS. 3B and 3C show in a side view the drilling template 41 in the pre-mounted state and after positioning upon the transverse surface 40 of the femur 34 respectively. The flexion of the knee joint is in said case corrected in such a way that the drilling template 41 and the surface 40 are movable into full mutual abutment. This is important for guaranteeing the correct positioning of the drill holes.

The drilling template 41 has two guides 42 for the drill as well as an insertion bore 43, into which in the next method step a feeler template 44 for determining the femur size is insertable.

FIG. 3D shows the resection height being checked once more by means of the scales 31, 32, 32′ prior to determination of the femur size by means of the feeler template 44 in order to ensure that later the correct section template is selected for the second femoral resection.

FIGS. 4A to 4C show the femur size being determined by sensing with a feeler template 44.

The feeler template 44 comprises an L-shaped bow 45, which has a scale 46 formed on the part of the bow 45 that is introduced into the insertion bore 43 of the drilling template 41. The length of the bow 45 in proximal-distal direction is variable by means of a displacement device 48.

On an opposite end of the feeler template 44 to the scale 46 a set-down part 47 is formed, which is set down onto the femur 34. By inserting the feeler template 44 into the insertion bore 43 until the set-down part 47 is set down on the femur 34, the femur size is determined and may be read off at the scale 46. In the embodiment, the scale 46 has five marks A, B, C, D and E that correspond to five different femur sizes, wherein A is the smallest size and E the largest size. The number of marks is not limited to five and may be higher or lower and/or have different distances between the marks. In the embodiment illustrated in FIG. 4C, the femur size has been determined by mark B. This is the mark that is still visible above the insertion bore 43 in the drilling template 44.

Through the guides 42 two drill holes 49 are now introduced into the surface 40 of the femur 34 and then the ligament-tensioning device 1 is removed. FIG. 5 shows the situation after introduction of the drill holes 49 and after removal of the ligament-tensioning device 1 in flexion. The two drill holes 49 have been introduced into the surface 40, the transversely oriented surfaces 36 and 40 are then parallel to one another in extension of the knee joint. FIG. 5 shows the initial situation for the last machining step of the preparation for supplying the implant, namely for the anterior and posterior resections of the femur 34.

According to the invention, for performing the remaining femoral resections only a single section template 50 is now required, which is shown by way of example in FIG. 6A. The section template 50 in this case is tuned to the size of the femur 34 previously determined by means of the feeler template 44, i.e. for each of the femur sizes A, B, C, D, E indicated on the scale 46 a separate section template 50 is provided, wherein the section templates 50 for the various femur sizes differ in their dimensions.

FIG. 6A shows by way of example the section templates 50 for a small femur 34 of size A (on the left in FIG. 6A) and for a large femur 34 of size E (on the right in FIG. 6A) in order to illustrate the differences.

The section template 50 in this case, irrespective of its dimensions, comprises a section block 51, which has two pins 52 for introduction into the drill holes 49 previously introduced into the transverse surface 40 of the femur 34. The pins 52 are in this case disposed approximately in the direction of a surface normal on the section block 51. The section block 51 further comprises saw blade guides 53, which are formed at different angles in the section block 51. The number of saw blade guides 53 in this case is four, which are set up for, in each case, a posterior femoral section, a posterior oblique section, an anterior oblique section and an anterior femoral section.

The section block 51 is in this case so designed that a distance X between a first saw blade guide 53a for a posterior femoral section, which in FIGS. 6A to 6D is in each case the lowermost (posterior) saw blade guide 53, and the pins 52 is of equal magnitude for all of the femur sizes A, B, C, D, E. This has the advantage that the posterior femoral section is effected always at the same point and so, later, the replacement knee joint may always be positioned correctly relative to the femur 34.

The saw blade guide 53a for the posterior femoral section is in this case of a two-part design, wherein the two partial slots are separated from one another by a web 54. This is advantageous for increasing the stability of the section block 51. The next saw blade guide 53b for a posterior oblique section is disposed inclined relative to the first saw blade guide 53a. The saw blade guide 53b for the posterior oblique section is likewise designed in two parts that are divided by the web 54.

A further saw blade guide 53c for an anterior oblique section is designed in the form of a fully enclosed slot and is inclined by approximately 90° relative to the saw blade guide 53b that is to be used for the posterior oblique section. A fourth saw blade guide 53d is likewise fully enclosed and is to be used to carry out the final anterior femoral section.

The two-part saw blade guides 53a and 53b are in this case so designed in terms of their dimensions as to allow reliable resection of the condyles 55 of the femur 34. The web 54 therefore has to be narrow enough to allow the complete resection. FIG. 6D shows in a side view a section template 50, in which the two saw blade guides 53a and 53b are visible from the side, while the two anterior saw blade guides 53c and 53d are visible only in projection in FIG. 6D because they are designed as fully enclosed slots.

In the embodiment two drill holes 56 are moreover formed in the section block 51 and are used to fix the section block 51 on the femur 34. Fixation nails may be driven through the drill holes 56 into the transverse surface 40 of the femur 34. This ensures that the section template 50 does not shift during the four resection cuts.

FIGS. 6B and 6C show the attaching of the section template 50 to the knee joint, which is still in flexion, and the section template 50 in situ respectively.

The fact that the section template has to be positioned only once and may then remain in situ for all of the four required femoral resections means, on the one hand, that handling of the section template 50 is made considerably easier for the surgeon. On the other hand, the machining accuracy is higher, with the result that follow-up resections may no longer apply, and the operating time may be considerably shortened because the section template 50 does not have to be re-positioned prior to each resection. This is advantageous particularly with regard to the use of navigation systems with electronic control because the calibration process of the navigation system is complex and, with the section template 50 according to the invention, need be carried out only once.

The invention is not limited to the illustrated embodiment, rather, given suitable adaptation, it is applicable also to ligament-tensioning devices 1 and section templates 50 for other spherical joints.

1. Ligament-tensioning device for preparing for the implantation of a joint implant having a basic body, which has first claws with first seating surfaces, which rest on a first bone part, and second claws, which rest with second seating surfaces on a second bone part, wherein the claws are displaceable relative to one another by means of parallel displacement devices, and having a first scale, which is disposed on the basic body of the ligament-tensioning device,
wherein second medial and lateral scales are provided, which correspond with the first scale, wherein the second scales are disposed on a guide body, which is displacement in proximal-distal direction relative to the basic body, in that a tension-lever-side lever and a basic-body-side lever are movable parallel to one another and/or away from one another, and wherein the scales indicate the planned resection heights medially and laterally on the second bone part while taking account of a ligament situation, and the height of an implant that is to be inserted into the joint to be treated is preadjustable medially and laterally separate from one another.

2. Ligament-tensioning device according to claim 1, wherein by means of the scales the height of an implant that is to be inserted into the joint to be treated is preadjustable.

3. Ligament-tensioning device according to claim 1, wherein the scales are disposed on the medial and lateral side of the guide body.

4. Ligament-tensioning device according to claim 1 wherein guide rods are formed in the guide body.

5. Ligament-tensioning device according to claim 4 wherein the guide rods are oriented in anteroposterior direction.

6. Ligament-tensioning device according to claim 4, wherein the guide rods are displaceable in anteroposterior direction in the guide body.

7. Ligament-tensioning device according to claim 4 wherein drilling templates are mountable onto the guide rods.

8. Ligament-tensioning device according to claim 7, wherein a position of the drilling templates relative to the second bone part is variable in two degrees of freedom by means of the ligament-tensioning device and the guide rods.

9. Method of preparing a joint for the implantation of a joint implant by means of a ligament-tensioning device, comprising a basic body, which has first claws with first seating surfaces, which rest on a first bone part, and second claws, which rest with second seating surfaces on a second bone part, wherein the claws are displaceable relative to one another by means of parallel displacement devices, and having a first scale, which is disposed on the basic body of the ligament-tensioning device, wherein second medial and lateral scales are disposed on a guide body, which is mounted displaceably relative to the basic body by means of the parallel displacement devices, and the scales are bringable into congruence in such a way that the height of an implant that is to be inserted into the joint to be treated is preadjustable medially and laterally separate from one another, having the following method steps:
perform a proximal tibial osteotomy;
perform a distal fermal osteotomy; and
carry out femoral oblique sections using a section template.

10. Method according to claim 9, wherein the joint implant is a knee joint implant that is implanted into the tibia and into the femur.

11. Method according to claim 9, wherein the second method step comprises at least one of the following sub-steps:
pre-mount a drilling template on the ligament-tensioning device,
introduce the ligament-tensioning device in the knee joint gap in extension position of the joint,
distract the ligament-tensioning device under a defined force,
adjust the desired thickness of the implant,
read off the distal resection length at the femur,
compare the values of the implant thickness and the resection length,
drill two holes into the femur through the drilling template,
place two pins into the femur,
remove the ligament-tensioning device from the knee joint gap,
bend the joint in flexion,
mount a section template onto the pins,
fix the section template on the femur by means of a fixation nail, and
perform the distal femoral osteotomy.

12. Method according to claim 11, wherein after the method step of comparing the values of the implant thickness and the resection length, in the event of deviations of the values, a tibial follow-up resection is performed or the implant thickness is increased.

13. Method according to claim 9, wherein the third method step comprises at least one of the following sub-steps:
pre-mount a drilling template on the ligament-tensioning device,
introduce the ligament-tensioning device into the knee joint gap,
distract the ligament-tensioning device under a defined force,
apply the drilling template against a transverse section surface of the femur,
drill two drill holes through the drilling template into the surface of the femur,
check the resection height using the scales,
measure the posterior projection of the femur by means of a feeler template,
determine the femur size,
select the section template, corresponding to the femur size,
relax and remove the ligament-tensioning device, and
perform the femoral resections by means of the section template.

14. Method according to claim 13, wherein performing the femoral resections by means of a section template comprises successively performing a posterior femoral section, a posterior oblique section, an anterior oblique section and an anterior femoral section.

15. Section template for femoral resection for preparing a joint for the implantation of a joint implant, wherein the section template comprises a section block, which has saw blade guides for performing resections of a femur, wherein the number of saw blade guides of the section template corresponds to the number of resections required for preparing the joint for the implantation of a joint implant.

16. Section template according to claim 15, wherein the joint implant is a femoral component of a replacement knee joint and the bone part to be resectioned is a femur.

17. Section template according to claim 15, wherein the section block rests against a previously resectioned, transversely oriented surface of the femur.

18. Section template according to claim 17, wherein the section block after being attached to the surface of the femur remains fixed on the femur until all of the resections have been performed.

19. Section template according to claim 18, wherein the number of saw blade guides is four.

20. Section template according to claim 19, wherein the four saw blade guides are provided for in each case a posterior femoral section, a posterior oblique section, a posterior oblique section, an anterior oblique section and an anterior femoral section.

21. Section template according to claim 15, wherein the section block is anchored by means of pins in specially provided drill holes of the femur.

22. Section template according to claim 20, wherein the posterior saw blade guides for the posterior resections are of a two-part design in the form of slots having a central web.

23. Section template according to claim 20, wherein the anterior saw blade guides for the anterior resections are of a one-piece slot-shaped design.

24. Section template according to claim 20, wherein the saw blade guides are disposed parallel to one another in anteroposterior direction.

25. Section template according to claim 20, wherein the saw blade guides provided for the oblique sections are disposed inclined at an angle of ca. 90° relative to one another.

26. Section template according to claim 20, wherein the saw blade guides provided for the straight fermoral sections are disposed approximately parallel to one another.

27. Section template according to claim 15 wherein the section block is fixed to the femur by means of fixation nails that are drivable into the femur through drill holes in the section block.
